(19) United States
(12) Patent Application Publication
(10) Pub. No.: US 2020/0318138 A1
(43) Pub. Date: Oct. 8, 2020

(54) CONSTRUCTS COMPRISING NEURONAL VIABILITY FACTORS AND USES THEREOF

(71) Applicants: SPARINGVISION, Paris (FR); INSERM (Institut National de la Santé et Recherche Médicale), Paris (FR); SORBONNE UNIVERSITE, Paris (FR)

(72) Inventors: Thierry LEVEILLARD, Maisons-Alfort (FR); Najate AÏT-ALI MAAMRI, Paris (FR); Fréderic BLOND, La Varenne Saint (FR); José-Alain SAHEL, Paris (FR); Géraldine PUEL, Thorigny Sur Marne (FR); Emmanuelle CLERIN, Grigny (FR)

(21) Appl. No.: 16/956,894

(22) PCT Filed: Dec. 21, 2018
(86) PCT No.: PCT/EP2018/086744

§ 371 Date: Jun. 22, 2020

(30) Foreign Application Priority Data
Dec. 22, 2017 (EP) 17306915.4

(51) Int. Cl. C12N 15/86; C07K 14/435

(52) U.S. Cl. CPC C12N 15/86; C12N 2830/008; C12N 2750/14143; C07K 14/435

(57) ABSTRACT

The present invention relates to improved constructs comprising the short and long Rod-Derived Cone Viability Factors and to methods for treating retinal degenerative diseases.

### FIELD OF THE INVENTION

The present invention relates to retinal neurodegenerative disorders, and more particularly to a pharmaceutical composition for treating and/or preventing neurodegenerative disorders.

### BACKGROUND OF THE INVENTION

Neurodegenerative disorder encompasses a range of seriously debilitating conditions that are characterized by neuron degeneration.

Rod-cone dystrophies, such as retinitis pigmentosa (RP), are genetically heterogeneous retinal degenerative diseases characterized by the progressive death of rod photoreceptors followed by the consecutive loss of cones. RP is one of the most common forms of inherited retinal degeneration, affecting around 1:3,500 people worldwide (1). Mutations causing RP in over 63 distinct genes have been identified to date with a significant proportion of these mutations in rod-specific transcripts. RP patients initially present with loss of vision under dim-light conditions as a result of rod dysfunction, with relative preservation of macular cone-mediated vision. As the disease progresses, however, the primary loss of rods is followed by cone degeneration, and a deficit in corresponding cone-mediated vision. In modern society, in which much of the environment is artificially lit, and many activities rely on high acuity color vision, retention of cone-mediated sight in RP patients would lead to a significant improvement in quality of life.

The loss of cones in RP subsets caused by rod-specific mutations is not perfectly understood, although several mechanisms, which are not necessarily mutually exclusive, have been proposed. Some hypothesized mechanisms implicate a ‘neighbor effect’ whereby cone death is a consequence of the release of endotoxins from the degeneration of surrounding rods, or as a result of the loss of contact with rods, retinal pigment epithelium (RPE) or Müller glia. Alternatively, activation of Müller cells and the release of toxic molecules may play a role. Another hypothesis is that the quantities of oxygen or retinoids delivered to the photoreceptor layer by the RPE from the choroidal blood circulation are excessive and toxic as the metabolic load of rods is lost (2). Punzo et al. showed evidence that in murine models of retinal degeneration cones die in part as a result of starvation and nutritional imbalance, driven by the insulin/mammalian target of rapamycin pathway (3). Additionally, it has been suggested that the loss of a survival factor secreted by rods and required for cone survival may contribute to cone loss (4, 5).

In agreement with the last hypothesis, transplanted healthy retinal tissue has been shown to support cone survival in areas distant from the grafted tissue in the rd1 mouse (6, 7).

International patent application WO2008/148860A1 describes a family of trophic factors, called rod-derived cone viability factor (RdCVF) and RdCVF2 that are able to increase neuron survival and are useful for treating and/or preventing neurodegenerative disorders such as RP.

The rod-derived cone viability factor (RdCVF) was originally identified from a high-throughput method of screening cDNA libraries as a candidate molecule responsible for this rescue effect (4). Rods secrete RdCVF, and therefore, as rods die, the source of this paracrine factor is lost and RdCVF levels decrease. The loss of expression of RdCVF, and secreted factors like it, may therefore contribute to the secondary wave of cone degeneration observed in rod-cone dystrophies. RdCVF has been shown to mediate cone survival both in culture (8) and when injected subretinally in mouse and rat models of recessive and dominant forms of retinitis pigmentosa (4, 9). Disruption of Nxnl1, the gene encoding RdCVF, renders mouse photoreceptors increasingly susceptible to photoreceptor dysfunction and cone loss over time (10).

Nxnl1 codes for two protein isoforms through differential splicing. The isoform mediating cone survival, RdCVF is a truncated thioredoxin-fold protein of its longer counterpart, RdCVFL, which includes a C-terminal extension conferring enzymatic thioloxidoreductase activity (11). RdCVFL, which contains all the amino acids of RdCVF, is encoded by exons 1 and 2 of the Nxnl1 gene and is a member of the thioredoxin family (12). Thioredoxins have diverse functions, including maintaining the proper reducing environment in cells and participating in apoptotic pathways. These functions are accomplished via thioloxidoreductase reactions mediated by a conserved CXXC catalytic site within a thioredoxin fold (13).

Byrne et al. (32) have shown that the two isoforms of RdCVF have complementary functions. Systemic administration of an adeno-associated virus (AAV) encoding RdCVF improved cone function and delayed cone loss, while RdCVFL increased rhodopsin mRNA and reduced oxidative stress. RdCVFL prevents photo-oxidative damage to the rods (36).

International patent application WO 2016/185037 describes AAV vectors encoding both RdCVF and RdCVFL, in particular the AAV CT35, and the use of said vectors for treating pathologies such as retinitis pigmentosa.

A synergistic effect between RdCVF and RdCVFL has been demonstrated (34). On the one hand, RdCVF is produced and secreted by the retinal pigmented epithelium (RPE), protecting the cones by stimulating aerobic glycolysis through the RdCVF receptor at the cell surface of the cones by a non-cell autonomous mechanism(37). On the other hand, RdCVFL, protects the cones against oxidative damage in a cell autonomous manner, due to its thioloxidoreductase function.

The inventors have now observed that the production of such AAV vectors unexpectedly presents a problem of encapsidation of the AAV genome. This problem of incomplete packaging leads to a defect in the production of AAV particles with full genome.

This presents a limitation for the production of GMP-compliant AAV vectors for use in human therapy.

Thus, there is still a need for improved constructs for the expression of RdCVF and RdCVFL factors for treating retinal neurodegenerative disorders.

### SUMMARY OF THE INVENTION

The inventors have found that the production of a single AAV vector comprising a first nucleic acid encoding RdCVF and a second nucleic acid encoding RdCVFL could be subject to problems of incomplete packaging of the AAV single strand DNA within the viral capsid. This incomplete encapsidation leads to the production of an incomplete AAV genome, so a DNA of a smaller size.

The inventors have surprisingly discovered that this incomplete encapsidation was due to the presence of direct repeated sequences within the AAV genome and that limiting the length of nucleotides that are identical between the first and second expression cassettes to at most 200 contiguous identical nucleotides, AAV can be fully packaged.

Thus, the present invention provides a solution to this problem, by providing AAV vectors comprising a first expression cassette comprising a first nucleic acid encoding RdCVF and a second expression cassette comprising a second nucleic acid encoding RdCVFL, in which production of AAV particles is optimized.

AAV according to the invention comprise a first and a second expression cassettes which display at most 200 contiguous identical nucleotides, preferably at most 190, even more preferably at most 180, 170, 167, 165, 164, 160, 150, 140, 130, 120, 110, 100, 90, 80, 70, 60, 55, 54, 50, 40, 30, 20, 15, 10, 9 or 8 contiguous identical nucleotides.

The inventors have developed several alternative and/or cumulative solutions to the problem of direct repeated sequences between the first and second expression cassettes.

Thus, in one aspect, the present invention relates to an adeno-associated vector (AAV) comprising:
a first expression cassette comprising a first nucleic acid encoding RdCVF and
a second expression cassette comprising a second nucleic acid encoding RdCVFL,
   wherein said first and second expression cassettes display less than 200 contiguous identical nucleotides.

In another aspect, the present invention relates to an adeno-associated vector (AAV) comprising:
a first expression cassette comprising a first nucleic acid encoding RdCVF and
a second expression cassette comprising a second nucleic acid encoding RdCVFL,
   for use in a method of treatment of a retinal neurodegenerative disorder,
   wherein said first and second expression cassettes display less than 200 contiguous identical nucleotides.

The present invention also relates to a method for treating a patient suffering from a retinal degenerative disease comprising the step consisting of administering to said patient a therapeutically effective amount of an adeno-associated vector (AAV) comprising:
a first expression cassette comprising a first nucleic acid encoding RdCVF and
a second expression cassette comprising a second nucleic acid encoding RdCVFL,
   wherein said first and second expression cassettes display less than 200 contiguous identical nucleotides.

The invention also relates to the use of an inert human DNA sequence in an AAV construct.

In one aspect, the invention relates to an AAV comprising a nucleic acid having the sequence set forth in SEQ ID NO: 10, wherein said nucleic acid having the sequence as set forth in SEQ ID NO: 10 is not present in an expression cassette.

### DETAILED DESCRIPTION OF THE INVENTION

Thus, in one aspect, the present invention relates to an adeno-associated vector (AAV) comprising:
a first expression cassette comprising a first nucleic acid encoding RdCVF and
a second expression cassette comprising a second nucleic acid encoding RdCVFL,
   wherein said first and second expression cassettes display less than 200 contiguous identical nucleotides.

The fact that the first and the second expression cassettes display less than 200 contiguous identical nucleotides means that the first and the second expression cassettes have less than 200 contiguous identical nucleotides in common.

Typically, the first and second expression cassettes share at most 200 contiguous identical nucleotides, preferably at most 190, even more preferably at most 180, 170, 167, 165, 164, 160, 150, 140, 130, 120, 110, 100, 90, 80, 70, 60, 55, 54, 50, 40, 30, 20, 15, 10, 9 or 8 contiguous identical nucleotides.

In another aspect, the present invention relates to an adeno-associated vector (AAV) comprising:
a first expression cassette comprising a first nucleic acid encoding RdCVF and
a second expression cassette comprising a second nucleic acid encoding RdCVFL,
   for use in a method of treatment of a retinal neurodegenerative disorder, wherein said first and second expression cassettes display less than 200 contiguous identical nucleotides.

The present invention also relates to a method for treating a patient suffering from a retinal degenerative disease comprising the step consisting of administering to said patient a therapeutically effective amount of an adeno-associated vector (AAV) comprising:
a first expression cassette comprising a first nucleic acid encoding RdCVF and
a second expression cassette comprising a second nucleic acid encoding RdCVFL,
   wherein said first and second expression cassettes display less than 200 contiguous identical nucleotides.

As used herein, the term Rod-derived Cone Viability Factor (RdCVF) refers to the protein encoded by the thioredoxin-like 6 (TXNL6) or Nucleoredoxin-like 1 (NXNL1) gene. It encompasses the RdCVF proteins of any animal species. Typically, the RdCVF proteins according to the present invention can be mammalian RdCVF proteins, including, but not limited to mice, rats, cats, dogs, non-human primates and human.

Unless otherwise specified, the term “RdCVF” refers to the short isoform of the NXNL1 gene and “RdCVFL” or ‘RdCVF-L” the long isoform of the NXNL1 gene.

Typically, in mice, the short isoform (RdCVF) is a 109 amino-acid long protein references under Uniprot accession number Q91W38. The murine long isoform (RdCVFL) is a 217 amino-acid long protein referenced under Q8VC33.

In one embodiment of the invention, the short isoform of RdCVF is the human short isoform of RdCVF (hRdCVF), having the following sequence:

```
(SEQ ID NO: 1)
                10 20 30 40 50
MASLFSGRIL IRNNSDQDEL DTEAEVSRRL ENRLVLLFFG AGACPQCQAF
                60 70 80 90 100
VPILKDFFVR LTDEFYVLRA AQLALVYVSQ DSTEEQQDLF LKDMPKKWLF
              109
LPFEDDLRR
```

Accordingly, the first nucleic acid, encoding the short isoform of RdCVF can comprise the following human nucleic acid sequence:

(SEQ ID NO: 3)
ATGGCCTCCCTGTTCTCTGGCCGCATCCTGATCCGCAACAATAGCGACCA
GGACGAGCTGGATACGGAGGCTGAGGTCAGTCGCAGGCTGGAGAACCGGC
TGGTGCTGCTGTTCTTTGGTGCTGGGGCTTGTCCACAGTGCCAGGCCTCT
CACAGATGAGTTCTATGTACTGCGGGCGGCTCAGCTGGCCCTGGTGTACG
TGTCCCAGTCGTGCCCATCCTCAAGGACTTCTTCGTGCGGGACTCCACGG
AGGAGCAGCAGGACCTGTTCCTCAAGGACATGCCAAAGAAATGGCTTTTC
CTGCCCTTTGAGGATGATCTGAGGAGGTGA

Alternatively, the first nucleic acid can comprise a nucleic acid which differs from SEQ ID NO: 3 but encodes the same amino acid sequence.

Suitable nucleic acid sequences include, but are not limited to:
polymorphisms of the cDNA encoding human RdCVF;
combinations of polymorphisms (rare haplotypes) of the cDNA encoding human RdCVF. An example of rare haplotype cDNA is set forth as SEQ ID NO: 11;
“optimized” sequences in which certain codons are replaced by codons that code for the same amino-acid. Suitable codon-optimized sequences encoding RdCVF include, but are not limited to, the sequence as set forth in SEQ ID NO: 12;
homologous sequences. For instance, the inventors have found that the chimpanzee cDNA sequence encoding the short isoform of chimpanzee RdCVF can be used, since it encodes the same amino acid sequence as the human cDNA. The chimpanzee cDNA has the sequence as set forth in SEQ ID NO: 4.

In one embodiment of the invention, the long isoform of the NXNL1 gene is the human long isoform RdCVFL (hRdCVFL), having the sequence referenced under accession number Q96CM4 and set forth below:

```
(SEQ ID NO: 2)
                10 20 30 40 50
MASLFSGRIL IRNNSDQDEL DTEAEVSRRL ENRLVLLFFG AGACPQCQAF
                60 70 80 90 100
VPILKDFFVR LTDEFYVLRA AQLALVYVSQ DSTEEQQDLF LKDMPKKWLF
              110 120 130 140 150
LPFEDDLRRD LGRQFSVERL PAVVVLKPDG DVLTRDGADE IQRLGTACFA
              160 170 180 190 200
NWQEAAEVLD RNFQLPEDLE DQEPRSLTEC LRRHKYRVEK AARGGRDPGG
              210
GGGEEGGAGG LF
```

Accordingly, the second nucleic acid, encoding RdCVFL can comprise the following human nucleic acid sequence:

(SEQ ID NO: 5)
ATGGCCTCCCTGTTCTCTGGCCGCATCCTGATCCGCAACAATAGCGACCA
GGACGAGCTGGATACGGAGGCTGAGGTCAGTCGCAGGCTGGAGAACCGGG
CTGGGGCTTGTCCACAGTGCCAGGCCTTCGTGCCCATCCTCAAGGACTTC
TCACAGATGAGTTCTATGTACTGCGGGCGGCTCAGCTGGCCCTGGTGTAC
GTGTCCCAGCTTCGTGCGGCTGGTGCTGCTGTTCTTTGGTGACTCCACGG
AGGAGCAGCAGGACCTGTTCCTCAAGGACATGCCAAAGAAATGGCTTTTC
CTGCCCTTTGAGGATGATCTGAGGAGGGACCTCGGGCGCCAGTTCTCAGT
GGAGCGCCTGCCGGCGGTCGTGGTGCTCAAGCCGGACGGGGACGTGCTCA
CTCGCGACGGCGCCGACGAGATCCAGCGCCTGGGCACCGCCTGCTTCGCC
AACTGGCAGGAGGCGGCCGAGGTGCTGGACCGCAACTTCCAGCTGCCAGA
GGACCTGGAGGACCAGGAGCCACGGAGCCTCACCGAGTGCCTGCGCCGCC
ACAAGTACCGCGTGGAAAAGGCGGCGCGAGGCGGGCGCGACCCCGGGGGA
GGGGGTGGGGAGGAGGGCGGGGCCGGGGGGCTGTTCTGA

Alternatively, the second nucleic acid can comprise a nucleic acid which differs from SEQ ID NO:5 but encodes the same amino acid sequence.

Suitable nucleic acid sequences include, but are not limited to:
polymorphisms of the cDNA encoding human RdCVF or combinations thereof;
“optimized” sequences in which certain codons are replaced by codons that code for the same amino-acid; Suitable codon-optimized sequences encoding RdCVF include, but are not limited to, the sequence as set forth in SEQ ID NO:12;
homologous sequences in other species.

The sequences of the RdCVF and RdCVFL proteins are described in Chalmel et al. 2007 (39) and in the international patent application WO2008/148860.

As used herein, the term “adeno-associated vector” or “AAV” has its general meaning in the art.

AAVs have been extensively described in the art as suitable vectors for gene delivery. Indeed, AAVs are non-pathogenic and display a broad range of tissue specificity, depending of their serotype. Typically, AAVs according to the present invention are AAVs that are able to target retinal cells.

Examples include, but are not limited to, AAV2, AAV8, AAV2/8, AAV2/5, AAV2/9, and AAV7m8.

In one embodiment, the AAV according to the present invention is obtained according to the method described in international patent application WO2012/158757.

Typically, the first and second nucleic acids, encoding respectively the short and long isoform of the NXNL1 gene, are under the control of a promoter that allows the expression of said short and long isoform in the target cells.

Suitable promoters can be ubiquitous promoters, such as the CMV/CBA promoter.

Suitable promoters can be promoters that enable the expression in the retina, preferably in retinal pigmented epithelial cells and photoreceptor cells.

In one embodiment, the promoter allows gene expression in retinal pigmented epithelial cells.

In one embodiment, the promoter allows gene expression in cone photoreceptors. A non-limiting example is the cone-opsin promoter.

Typically, the short isoform of the NXNL1 gene is expressed at least by retinal pigmented epithelial cells and the long isoform is expressed at least by cone photoreceptor cells.

In one embodiment of the invention, different promoters are used to drive the expression of the short isoform and of the long isoform.

Typically, the short isoform of the NXNL1 gene can be expressed under the control of the CMV/CBA promoter and the long isoform of the NXNL1 gene can be expressed under the control of the cone-opsin promoter.

In one embodiment of the invention, the two expression cassettes are inverted, with one expression cassette being 5′ to 3′ and the other expression cassette being 3′ to 5′.

The inventors have found that this configuration was also suitable to avoid incomplete packaging.

In one embodiment of the invention, said adeno-associated vector (AAV) above described further comprises a stuffer sequence of SEQ ID NO: 10.

In a specific embodiment, the present invention relates an adeno-associated vector (AAV) comprising:
a first expression cassette comprising a first nucleic acid comprising a codon-optimized cDNA encoding RdCVF, under the control of an ubiquitous promoter, preferably the CMV/CBA promoter,

and
a second expression cassette comprising a second nucleic acid comprising a codon-optimized cDNA encoding RdCVFL under the control of the cone-opsin promoter (OPN1L/MW).

In one embodiment, the first nucleic acid comprising a codon-optimized cDNA encoding RdCVF has the sequence set forth in SEQ ID NO: 12.

In one embodiment, the second nucleic acid comprising a codon-optimized cDNA encoding RdCVFL has the sequence set forth in SEQ ID NO: 13.

In one embodiment the adeno-associated vector has the sequence as set forth in SEQ ID NO: 6 (corresponding to construct 3 of the Examples below).

In the context of the invention, the term “treating” or “treatment”, as used herein, means reversing, alleviating, inhibiting the progress of, or preventing the disorder or condition to which such term applies, or one or more symptoms of such disorder or condition (e.g., retinal degenerative diseases).

The term “retinal degenerative diseases” encompasses all diseases associated with cone degeneration. retinal degenerative disease include but are not limited to Retinitis Pigmentosa, age-related macular degeneration, Bardet-Biedel syndrome, Bassen-Kornzweig syndrome, Best disease, choroidema, gyrate atrophy, Leber congenital amaurosis, Refsum disease, Stargardt disease or Usher syndrome.

In one embodiment of the invention, the retinal degenerative disease is Retinitis Pigmentosa.

According to the invention, the term “patient” or “patient in need thereof” is intended for a human or non-human mammal affected or likely to be affected with retinal degenerative diseases.

According to the present invention, a “therapeutically effective amount” of a composition is one which is sufficient to achieve a desired biological effect, in this case increasing the neuron viability. It is understood that the effective dosage will be dependent upon the age, sex, health, and weight of the recipient, kind of concurrent treatment, if any, frequency of treatment, and the nature of the effect desired. However, the preferred dosage can be tailored to the individual subject, as is understood and determinable by one of skill in the art, without undue experimentation.

The expression vector of the invention can be suitable for intraocular administration. In a particular embodiment, the expression vector is administered by sub-retinal injection.

In one aspect, the invention also relates to a pharmaceutical composition comprising an adeno-associated vector (AAV) and a pharmaceutically acceptable carrier, wherein said AAV comprises:
a first expression cassette comprising a first nucleic acid encoding RdCVF
a second expression cassette comprising a second nucleic acid encoding RdCVFL,
   wherein said first and second expression cassettes display less than 200 contiguous identical nucleotides.

In another aspect, the invention relates to an AAV comprising a nucleic acid having the sequence set forth in SEQ ID NO:10, wherein said nucleic acid having the sequence as set forth in SEQ ID NO:10 is not present in an expression cassette. This sequence SEQ ID NO: 10 has the role of stuffer DNA in the AAV. The role of a stuffer sequence is to increase the size of the vector in order to avoid that the proviral plasmid is encapsidated instead of the gene of interest.

Usually, stuffers corresponding to bacteriophage lambda DNA are used in AAV. However, the sequences of bacteriophage lambda most commonly used as stuffer contain open reading frames, the nin regions. Although there are considered as inert because of phylogenetic distance to Human, Cheng et al. (38) have demonstrated that such stuffer were not as inert as expected because the nin regions may have strong transcription activity. Thus, when AAV with such stuffer are administrated to a human patient, there is a risk of transcription of the bacteriophage lambda DNA.

Thus, it was important to develop a new inert stuffer. The inventors have found that the nucleic acid having the sequence as set forth in SEQ ID NO: 10 could unexpectedly be used as a “stuffer” DNA, in order to increase the size of the AAV constructs, in replacement of the bacteriophage lambda stuffer. Said nucleic acid having the sequence as set forth in SEQ ID NO:10 has the great advantage of being inert because this sequence is a non-translated sequence, is not a miRNA target, is a non-telomeric sequence, it does not contain any origin of DNA replication and contains no nucleotides repeat. This sequence having any functional activity, there is not risk of transcription when it is used as a stuffer in an AAV.

The sequence as set forth in SEQ ID NO: 10 was selected through a thorough process as described in FIG. 3. It is a bioinformatic approach which consists of identifying in the human genome region that does not contains any elements as centromeres, genes, pseudo genes, replication origins, micro RNA targeted sequences or repeats. The sequence NO: 10 was selected among these loci.

The invention will be further illustrated through the following examples and figures.

### FIGURES LEGENDS

FIG. 1: Schematic representation of preferred constructs according to the invention:

FIG. 1A represents the construct CT35, a comparative example (thus not a construct according to the present invention) disclosed in WO2016/185037. FIGS. 1B, 1C and 1D respectively represent constructs 3 (C03), 6 (C06) and 11 (C11) according to the invention.

FIG. 2: Single strand DNA of AAV genome size analyzed by denaturing gel electrophoresis

The size of the single strand DNA of the AAV genome of different constructs was analyzed by gel electrophoresis under denaturing conditions:
Construct 3 (C03) which expresses both RdCVF and RdCVFL (7^{v7}. AAV2-CMV/CBA^{orig}-RdCVF-5′_1.7.OPN1L/MW-RdCVFL)
CT35 which expresses both RdCVF and RdCVFL (AAV2-CMV/CBA-RdCVF-CMV/CBA-RdCVFL) (SEQ ID NO: 15)
CT37 which only expresses RdCVF (AAV2-CMV/CBA-RdCVF+stuffer) (SEQ ID NO: 16).

FIG. 3: Schematic representation of the selection process for an inert “stuffer” DNA

FIG. 4: Packaging comparison

FIG. 4A: Other representation of the AAV CT35. In this AAV expressing both RdCVF and RdCVFL, the first and the second expression cassettes have 390 contiguous identical nucleotides due to the direct repeat of the promoter [CMV/CBA delta 390].

FIG. 4B: Graphical simulation of a recombination between the two copies [CMV/CBA delta 390] which would produce an elimination of the cassette [CMV/CBA delta 390-RdCVF] or a recombination with the cassette [CMV/CBA delta 390-RdCVFL].

FIG. 4C: Transduction of CT35 (AAV-CMV/CBA-RdCVF_CMV/CBA-RdCVFL) in primary cells of porcine pigmented epithelium. Western blot analysis of RdCVF and RdCVFL using rabbit polyclonal anti-RdCVF antibodies (4).

FIG. 4D: Other representation of the AAV C06 and graphical simulation of a recombination. In this AAV, the first and the second expression cassettes share a direct repeat of 167 contiguous identical nucleotides.

FIG. 4E: Other representation of the AAV C03.

FIG. 4F: Genome integrity analysis of encapsidated AAVs C06, C03, CT35 and CT37 (capsid proteins plus DNA).

FIGS. 4G and 4H: Tables representing for C03 and C06 the percentage of capsids comprising a complete encapsidated AAV (full), the percentage of capsids comprising an incompletely encapsidated AAV (intermediate) and the percentage of capsids comprising no AAV (empty; without DNA). Table 4G shows detection results obtained for both capsid protein and DNA (AAV genome). Table 4H shows detection results obtained only for DNA.

### EXAMPLES

### Example 1

The following section provides non-limiting examples of suitable constructs according to the invention.

Construct 3 (C03): AAV2-CMV/CBA^{orig}-RdCVF-5′_1.7.OPN1L/MW-RdCVFL

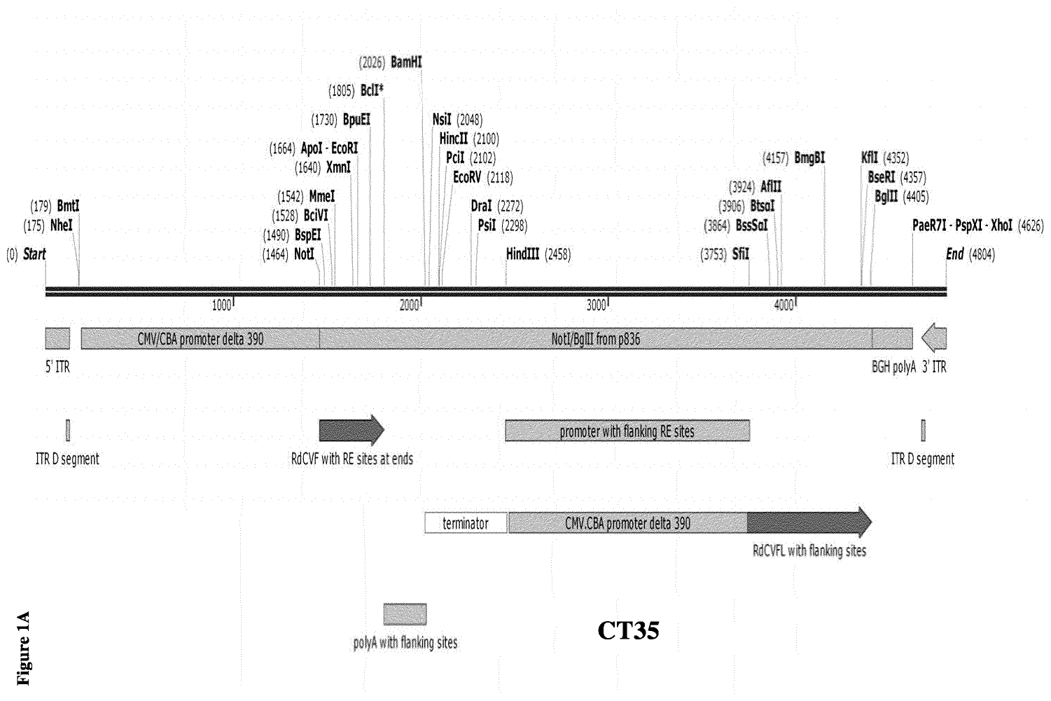

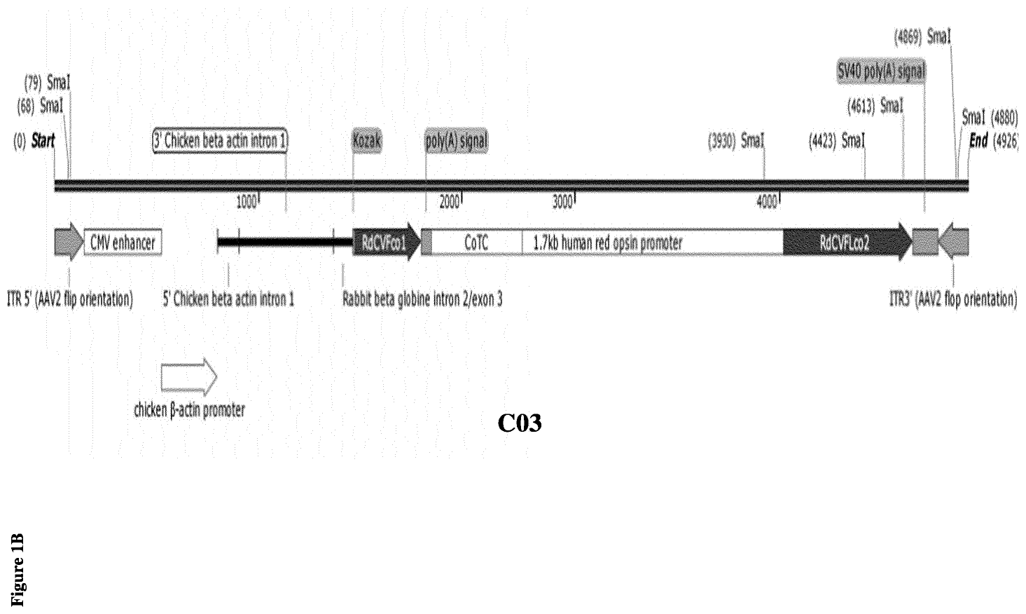

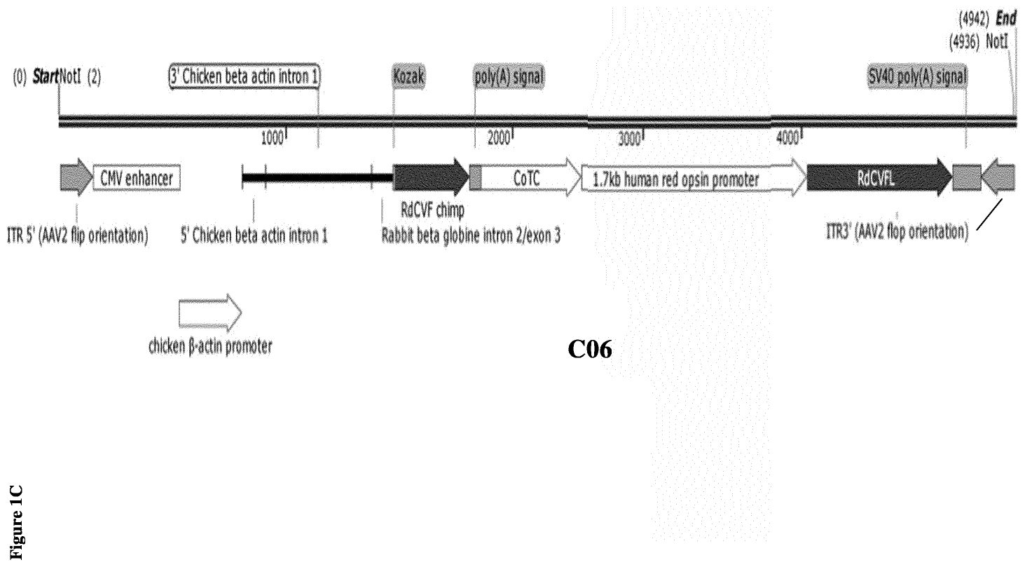

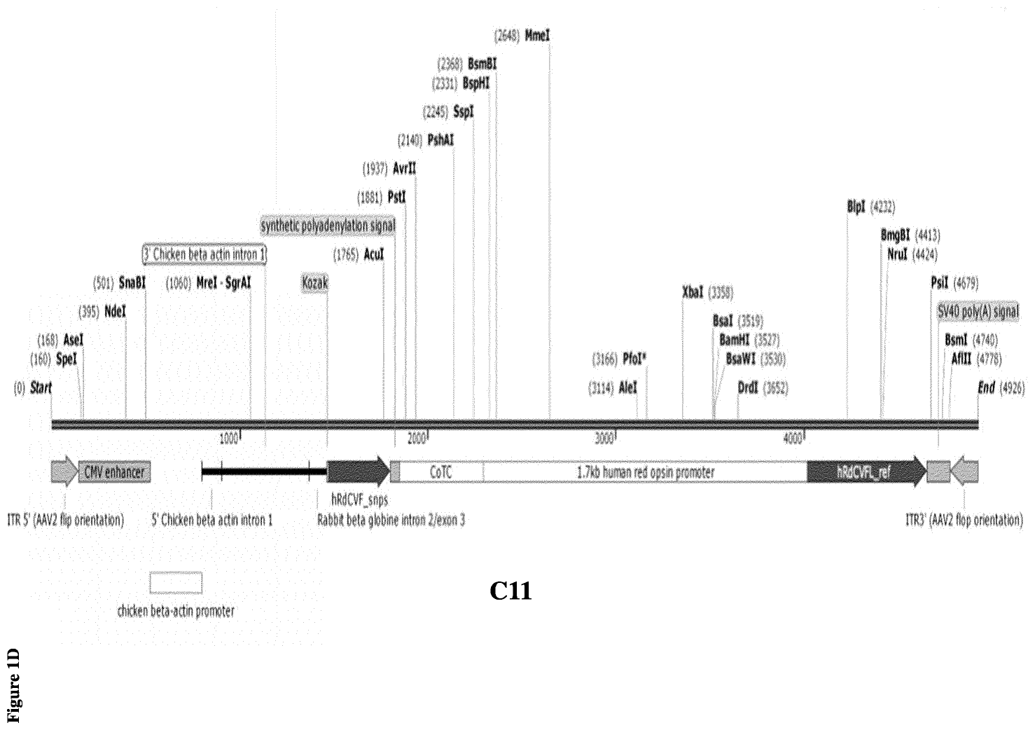

As shown on FIG. 1B, in this construct, the human RdCVF cDNA sequence was codon optimized (using a first optimization process v1) and placed under the ubiquitous promoter CMV/CBA. The human RdCVFL cDNA was also codon optimized (using a different optimization process v2) and was placed under the control of the cone-opsin promoter. The direct repeat shared between the first and the second expression cassette is 9 nucleotides long.

The AAV vector has the sequence as set forth in SEQ ID NO: 6.

Construct 6 (C06):

AAV2_CMV/CBA_orig_RdCVF_chimp_5p_1.7_OPN1LMW_RdCVFL

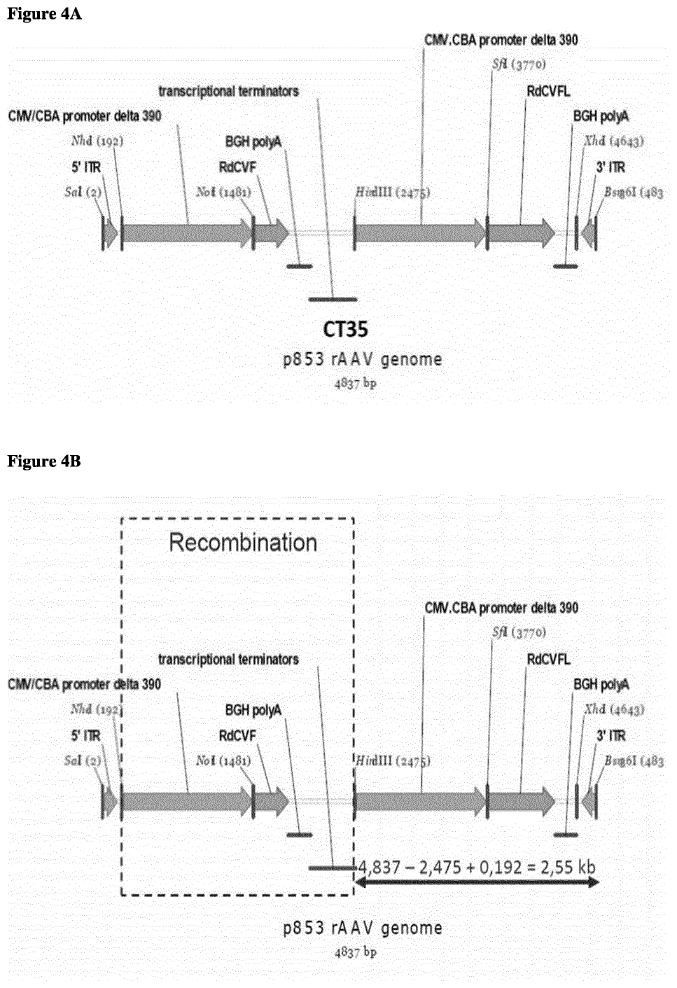

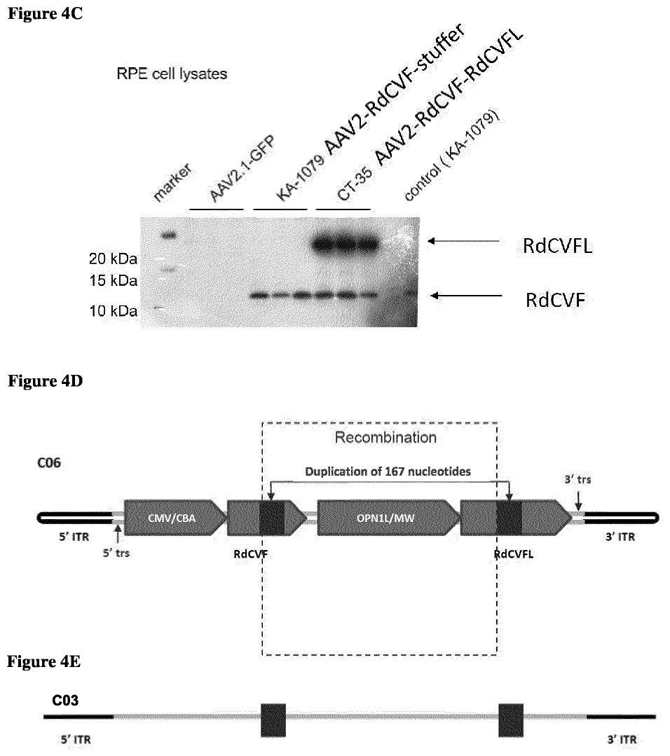

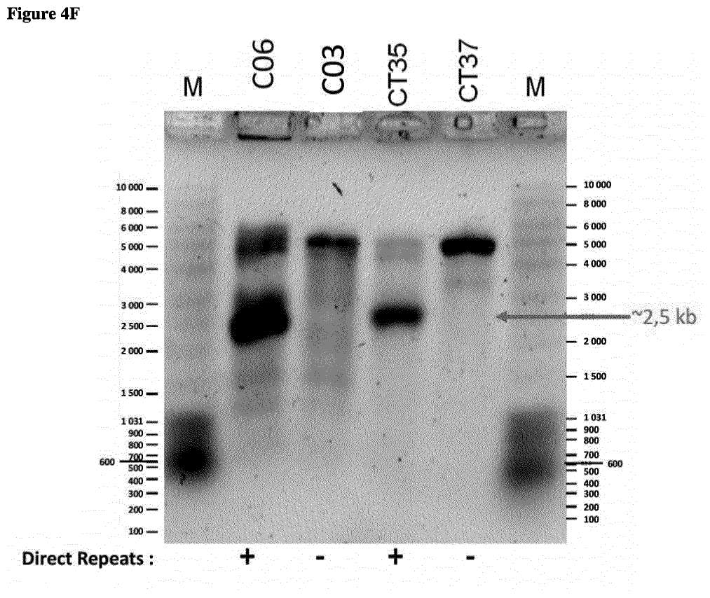

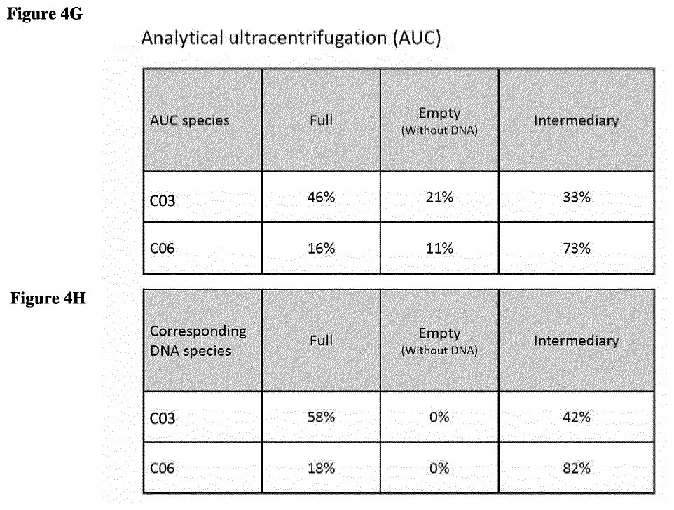

As shown on FIGS. 1C and 4D, in this construct, the chimpanzee RdCVF cDNA sequence was used in the first expressed cassette and placed under the ubiquitous promoter CMV/CBA. The human RdCVFL cDNA was placed under the control of the cone-opsin promoter. The direct repeat shared between the first and the second expression cassette is 167 nucleotides long.

The AAV vector has the sequence as set forth in SEQ ID NO: 7.

Construct 7 (C07):

AAV2_rev_CMV/CBA_orig_RdCVF_chimp_5p_1.7_OPN1LMW_RdCVFL

This construct is similar to construct 6, except that the first expression cassette is placed in reverse orientation.

The AAV vector has the sequence as set forth in SEQ ID NO: 8.

Construct 8 (C08):

AAV2_CMV/CBA_orig_RdCVF_chimp_rev_5p_1.7_OPN1LMW_RdCVFL-hGH

This construct is similar to construct 6, except that the second expression cassette is placed in reverse orientation.

The AAV vector has the sequence as set forth in SEQ ID NO: 9.

Construct 11 (C11):

AAV2_CMV/CBA_orig_RdCVF_rare_haplotype_human_5p_1.7_OPN1LMW_RdCVF L

As shown on FIG. 1D, in this construct, the first expression cassette comprises a cDNA encoding human RdCVF that is a combination of polymorphisms (rare haplotype), under the ubiquitous promoter CMV/CBA. The second expression cassette comprises the human RdCVFL cDNA, under the control of the cone-opsin promoter. The direct repeat shared between the first and the second expression cassette is 54 nucleotides long.

The AAV vector has the sequence as set forth in SEQ ID NO: 14.

### Example 2: AAV Constructs Displaying Long Stretches of Identical Nucleotides are Subject to Incomplete Packaging

### Material and Methods

### Production of Viral Vectors

AAV vectors carrying cDNA encoding mouse-RdCVF, RdCVFL or eGFP were produced by the plasmid co-transfection method (31). Recombinant AAV was purified by cesium chloride or iodixanol gradient ultracentrifugation. The viral eluent was buffer exchanged and concentrated with Amicon Ultra-15 Centrifugal Filter Units in PBS and titrated by quantitative PCR relative to a standard curve.

### Denaturing Gel Electrophoresis

The genomic DNA was extracted and subjected to denaturing gel electrophoresis. The size of the nucleic acids was compared to a DNA ladder.

### Results

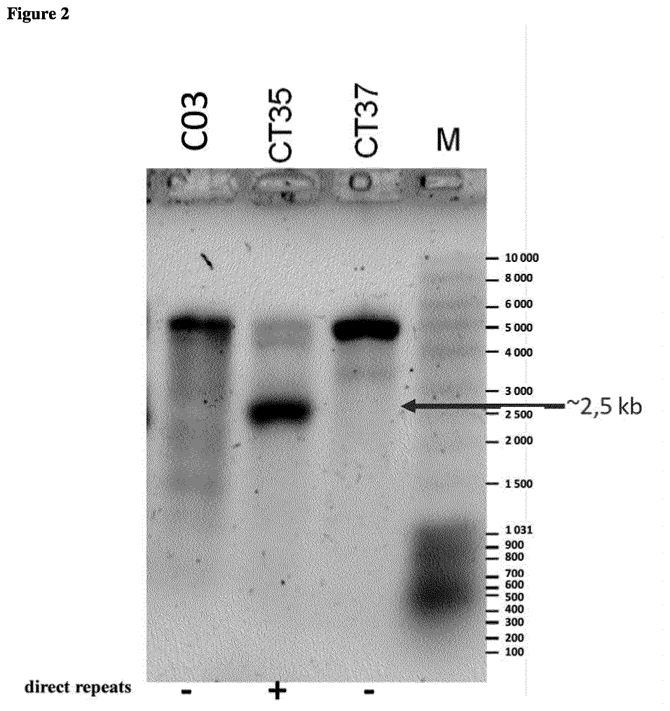

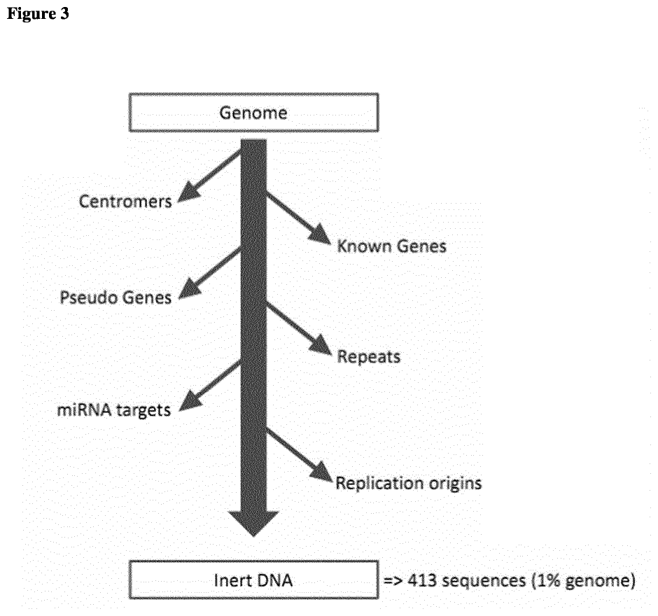

The FIGS. 2 and 4F show that the construct CT37, disclosed in WO2016/185037 and thus not a construction according to the invention. This comparative example which only expresses RdCVF is subject to a complete packaging since its production results in DNA at the expected sizes of ˜5000 bp.

As shown at FIGS. 2 and 4F, CT35, which comprises a repeat of 390 contiguous identical nucleotides (FIG. 4A), is subject to incomplete packaging since its production results in abnormal AAV genome sizes instead of AAV genome of 4804 bp.

Thus, it is well demonstrated that expressing a first nucleic acid encoding RdCVF and a second nucleic acid encoding RdCVFL in an AAV can lead to incomplete encapsidation of the AAV single strand DNA within the viral capsid.

In contrast, the construct C06 according to the invention, which expresses both RdCVF and RdCVFL and comprises a direct repeat of only 167 nucleotides long, show a band at the expected size of 4942 bp. This result demonstrates a complete encapsidation with the construct C06.

In the same way, the construct C03, which comprises a repeat of 9 contiguous identical nucleotides, shows a single band at the expected size of 4926 bp.

It is shown that decreasing the number of contiguous identical nucleotides shared between the two expression cassettes, allows to increase the proportion of complete encapsidation of an AAV comprising both a nucleic acid encoding RdCVF and a nucleic acid encoding RdCVFL.

Thus, the inventors have demonstrated that complete packaging is obtained when the first and second expression cassette do not contain more than 200 contiguous nucleic acids.

To further explore the phenomenon, analytical ultracentrifugation was used according to Burnham et al. (35) to compare the different constructs (FIGS. 4G and 4H). The results for C03 and C06 show that the extra band observed in the denaturing gel matches that of the high percentage of AAV particles with intermediary sedimentation coefficients, represent particles that are between full and empty, and thus corresponding to particles comprising an AAV incompletely packaged. In accordance with the results obtained in the denaturing gel electrophoresis, construct C06 shows full encapsidation of 18% and construct C03 yielded appropriate results in the analytical ultracentrifugation method, indicating a high percentage of full AAV particles (58%) (FIG. 4H).

This confirms that there is an increase of the percentage of particles with integral genome when the number of contiguous identical nucleotides shared between the two expression cassettes is no superior to 200.

### Example 3: Combination of RdCVF and RdCVFL Results in a Synergistic Effect

The following constructs have been produced and introduced into an AAV2 vector.

The proviral plasmid p618 and its elements are described in international patent application published as WO2012158757A1 and in publication (33).

2xRdCVF: Plasmid p857 and AAV CT39

P857/CT39 was designed to increase the level of expression of RdCVF as compared to CT37 (RdCVF-stuffer) to achieved sufficient cone protection in patients suffering from retinitis pigmentosa (RP).

RdCVF-RdCVFL: Plasmid p853 and AAV CT35

This vector is able to co-express the short and long isoform of RdCVF.

However, its production is subject to abnormal incomplete packaging events which limit its use as a therapeutic agent.

### Example 4: Selection of an Inert DNA for Replacing Phage Lambda Stuffers

The inventors have developed a screening process for identifying a nucleic acid sequence which could be used as a safer alternative to the phage lambda stuffer sequences traditionally used in order to obtain AAV constructs having a sufficient size.

Screening of the entire human genome was performed in order to eliminate undesirable sequences such as centromers, known genes, pseudogenes, repeats, miRNA targets, replication origins. This inventive screening process resulted in the selection of SEQ ID NO: 10, which is an inert sequence from human chromosome 15.

### Example 5: Recombination Analysis

Western blot analysis at FIG. 4C shows the expression of RdCVF and RdCVFL using rabbit polyclonal anti-RdCVF antibodies. Both proteins are detected for the construct CT35, which demonstrates that CT35 is not subject to homologue recombination.

### REFERENCES

Throughout this application, various references describe the state of the art to which this invention pertains. The disclosures of these references are hereby incorporated by reference into the present disclosure.
1. Buch H et al. Prevalence and causes of visual impairment and blindness among 9980 Scandinavian adults: the Copenhagen City Eye Study. Ophthalmology. 2004; 111(1):53-61.
2. Bramall A N, Wright A F, Jacobson S G, McInnes R R. The genomic, biochemical, and cellular responses of the retina in inherited photoreceptor degenerations and prospects for the treatment of these disorders. Annu Rev Neurosci. 2010; 33(1):441-472.
3. Punzo C, Kornacker K, Cepko C L. Stimulation of the insulin/mTOR pathway delays cone death in a mouse model of retinitis pigmentosa. Nat Neurosci. 2009; 12(1):44-52.
4. Léveillard T et al. Identification and characterization of rod-derived cone viability factor. Nat Genet. 2004; 36(7):755-759.
5. Mohand-Said S et al. Normal retina releases a diffusible factor stimulating cone survival in the retinal degeneration mouse. Proc Natl Acad Sci USA. 1998; 95(14):8357-8362.
6. Mohand-Said S et al. Photoreceptor transplants increase host cone survival in the retinal degeneration (rd) mouse. Ophthalmic Res. 1997; 29(5):290-297.
7. Mohand-Said S, Hicks D, Dreyfus H, Sahel J-A. Selective transplantation of rods delays cone loss in a retinitis pigmentosa model. Arch Ophthalmol. 2000; 118(6):807-811.
8. Wang X W, Tan B Z, Sun M, Ho B, Ding J L. Thioredoxin-like 6 protects retinal cell line from photooxidative damage by upregulating NF-kappaB activity. Free Radic Biol Med. 2008; 45(3):336-344.
9. Yang Y et al. Functional Cone Rescue by RdCVF Protein in a Dominant Model of Retinitis Pigmentosa. Mol Ther. 2009; 17(5):787-795.
10. Cronin T et al. The disruption of the rod-derived cone viability gene leads to photoreceptor dysfunction and susceptibility to oxidative stress. Cell Death Differ. 2010; 17(7):1199-1210.
11. Brennan L A, Lee W, Kantorow M. TXNL6 is a novel oxidative stress-induced reducing system for methionine sulfoxide reductase a repair of α-crystallin and cytochrome C in the eye lens. PLoS ONE. [published online ahead of print: 2010]; doi:10.1371/journal.pone.0015421.g008
12. Funato Y, Mild H. Nucleoredoxin, a Novel Thioredoxin Family Member Involved in Cell Growth and Differentiation. Antioxid Redox Signal. 2007; 9(8):1035-1058.
13. Lillig C H, Holmgren A. Thioredoxin and Related Molecules—From Biology to Health and Disease. Antioxid Redox Signal. 2007; 9(1):25-47.
14. Barhoum R et al. Functional and structural modifications during retinal degeneration in the rd10 mouse. Neuroscience. 2008; 155(3):698-713.
15. Phillips M J, Otteson D C, Sherry D M. Progression of neuronal and synaptic remodeling in the rd10mouse model of retinitis pigmentosa. J Comp Neurol. 2010; 518(11):2071-2089.
16. Gargini C et al. Retinal organization in the retinal degeneration 10 (rd10) mutant mouse: A morphological and ERG study. J Comp Neurol. 2006; 500(2):222-238.
17. Pang J J et al. AAV-mediated gene therapy for retinal degeneration in the rd10 mouse containing a recessive PDEbeta mutation. Investigative Ophthalmology & Visual Science. 2008; 49(10):4278-4283.
18. Pang J J et al. Long-term retinal function and structure rescue using capsid mutant AAV8 vector in the rd10 mouse, a model of recessive retinitis pigmentosa. Mol Ther. 2011; 19(2):234-242.
19. Komeima K, Rogers B S, Campochiaro P A. Antioxidants slow photoreceptor cell death in mouse models of retinitis pigmentosa. J Cell Physiol. 2007; 213(3):809-815.
20. Dalkara D et al. In vivo-directed evolution of a new adeno-associated virus for therapeutic outer retinal gene delivery from the vitreous. Science Translational Medicine. 2013; 5(189):189ra76.
21. Dalkara D et al. Enhanced gene delivery to the neonatal retina through systemic administration of tyrosine-mutated AAV9. Gene Ther. 2012; 19(2):176-181.
22. Cao W, Wen R, Li F, Lavail M M, Steinberg R H. Mechanical injury increases bFGF and CNTF mRNA expression in the mouse retina. Experimental Eye Research. 1997; 65(2):241-248.
23. Hollander den A I, Black A, Bennett J, Cremers F P M. Lighting a candle in the dark: advances in genetics and gene therapy of recessive retinal dystrophies. J Clin Invest. 2010; 120(9):3042-3053.
24. Maguire A M et al. Safety and Efficacy of Gene Transfer for Leber's Congenital Amaurosis. N Engl J Med. 2008; 358(21):2240-2248.
25. Cideciyan A V et al. Human gene therapy for RPE65 isomerase deficiency activates the retinoid cycle of vision but with slow rod kinetics. Proceedings of the National Academy of Sciences. 2008; 105(39):15112-15117.
26. Bainbridge J W B et al. Effect of Gene Therapy on Visual Function in Leber's Congenital Amaurosis. N Engl J Med. 2008; 358(21):2231-2239.
27. Fridlich R et al. The Thioredoxin-like Protein Rod-derived Cone Viability Factor (RdCVFL) Interacts with TAU and Inhibits Its Phosphorylation in the Retina. Molecular & Cellular Proteomics. 2009; 8(6): 1206-1218.
28. Mingozzi F et al. CD8(+) T-cell responses to adeno-associated virus capsid in humans. Nat Med. 2007; 13(4):419-422.
29. Manno C S et al. Successful transduction of liver in hemophilia by AAV-Factor IX and limitations imposed by the host immune response. Nat Med. 2006; 12(3):342-347.
30. Jacobson S G et al. Gene therapy for leber congenital amaurosis caused by RPE65 mutations: safety and efficacy in 15 children and adults followed up to 3 years. Arch Ophthalmol. 2012; 130(1):9-24.
31. Grieger J C, Choi V W, Samulski R J. Production and characterization of adeno-associated viral vectors. Nat Protoc. 2006; 1(3):1412-1428.
32. Byrne L C, Dalkara D, Luna G, Fisher S K, Clérin E, Sahel J A, Léveillard T, Flannery J G. Viral-mediated RdCVF and RdCVFL expression protects cone and rod photoreceptors in retinal degeneration. J Clin Invest. 2015 125(1):105-16.
33. Vasireddy, V., Mills, J. A., Gaddameedi, R., Basner-Tschakarjan, E., Kohnke, M., Black, A. H., Alexandrov, K., Maguire, A. M., Chung, D. C., Mac, H., Sullivan, L., Gadue, P., Bennicelli, J. L., French, D. L., and Bennett, J. AAV-mediated gene therapy for choroideremia: Preclinical studies in personalized models. PLoS ONE January 2013; 8(5):e61396.
34. Mei X., Chaffiol, A., Kole, C., Yang Y., Millet-Puel G., Clérin E., Aït-Ali N., Bennett, J., Dalkara D., Sahel J A, Duebel, J., Léveillard T., The thioredoxin encoded by the Rod-derived Cone Viability Factor gene protects cone photoreceptors against oxidative stress. Antioxid Redox Signal. 2016 May 12. [Epub ahead of print].
35. Burnham B, Nass S, Kong E, Mattingly M, Woodcock D, Song A, Wadsworth S, Cheng S H, Scaria A, O'Riordan C R: Analytical Ultracentrifugation as an Approach to Characterize Recombinant Adeno-Associated Viral Vectors. Human gene therapy methods 2015, 26(6):228-242.
36. Elachouri G, Lee-Rivera I, Clerin E, Argentini M, Fridlich R, Blond F, Ferracane V, Yang Y, Raffelsberger W, Wan J, Bennett J, Sahel J A, Zack D J, Leveillard T: Thioredoxin rod-derived cone viability factor protects against photooxidative retinal damage. Free radical biology & medicine 2015, 81:22-29.
37. Ait-Ali N, Fridlich R, Millet-Puel G, Clerin E, Delalande F, Jaillard C, Blond F, Perrocheau L, Reichman S, Byrne L C, Olivier-Bandini A, Bellalou J, Moyse E, Bouillaud F, Nicol X, Dalkara D, van Dorsselaer A, Sahel J A, Leveillard T: Rod-derived cone viability factor promotes cone survival by stimulating aerobic glycolysis. Cell 2015, 161(4):817-832.
38. Cheng S W, Court D L, Friedman D I: Transcription termination signals in the nin region of bacteriophage lambda: identification of Rho-dependent termination regions. Genetics 1995, 140(3):875-887.
39. Chalmel, F., Leveillard, T., Jaillard, C., Lardenois, A., Berdugo, N., Morel, E., Koehl, P., Lambrou, G., Holmgren, A., Sahel, J. A., and Poch, O. (2007). Rod-derived Cone Viability Factor-2 is a novel bifunctional-thioredoxin-like protein with therapeutic potential. BMC molecular biology 8, 74.

1. An adeno-associated vector (AAV) comprising:
a first expression cassette comprising a first nucleic acid encoding RdCVF
and
a second expression cassette comprising a second nucleic acid encoding RdCVFL,
wherein said first and second expression cassettes display less than 200 contiguous identical nucleotides.

2. The AAV according to claim 1, wherein the AAV is a serotype AAV2/8.

3. The AAV according to claim 1, wherein the first nucleic acid encoding RdCVF is under the control of an ubiquitous promoter.

4. The AAV according to claim 1, wherein the second nucleic acid encoding RdCVFL is under the control of the cone-opsin promoter.

5. The AAV according to claim 1, wherein the AAV has a nucleic acid sequence as set forth in a sequence selected from the group consisting of SEQ ID NO: 6, SEQ ID NO: 7, SEQ ID NO: 8 and SEQ ID NO: 9.

6. The AAV according to claim 1, wherein the AAV further comprises a stuffer sequence of SEQ ID NO:10.

7. (canceled)

8. A method for treating a patient suffering from a retinal degenerative disease consisting of administering to said patient a therapeutically effective amount of an adeno-associated vector (AAV) comprising:
a first expression cassette comprising a first nucleic acid encoding RdCVF
and
a second expression cassette comprising a second nucleic acid encoding RdCVFL,
wherein said first and second expression cassettes display less than 200 contiguous identical nucleotides.

9. An AAV comprising a nucleic acid having the sequence set forth in SEQ ID NO:10, wherein said nucleic acid having the sequence as set forth in SEQ ID NO:10 is not present in an expression cassette.

10. The AAV according to claim 3, wherein the ubiquitous promoter is a CMV/CBA promoter.

11. The AAV according to claim 5, wherein the AAV has a nucleic acid sequence as set forth SEQ ID NO: 6.


SEQUENCE LISTING (16 sequences)

SEQ ID NO: 1 - protein, 109 aa, Homo sapiens
MASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRR

SEQ ID NO: 2 - protein, 212 aa, Homo sapiens
MASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRRDLGRQFSVERLPAVVVLKPDGDVLTRDGADEIQRLGTACFANWQEAAEVLDRNFQLPEDLEDQEPRSLTECLRRHKYRVEKAARGGRDPGGGGGEEGGAGGLF

SEQ ID NO: 3 - DNA, 330 bp, Homo sapiens
atggcctccctgttctctggccgcatcctgatccgcaacaatagcgaccaggacgagctggatacggaggctgaggtcagtcgcaggctggagaaccggctggtgctgctgttctttggtgctggggcttgtccacagtgccaggccttcgtgcccatcctcaaggacttcttcgtgcggctcacagatgagttctatgtactgcgggcggctcagctggccctggtgtacgtgtcccaggactccacggaggagcagcaggacctgttcctcaaggacatgccaaagaaatggcttttcctgccctttgaggatgatctgaggaggtga

SEQ ID NO: 4 - DNA, 330 bp, Pan troglodytes
atggcctccctgttctctggccgcatcctgatccgcaacaatagcgaccaggacgaactggatacggaggctgaggtcagtcgcaggctggagaaccggctggtgctgctgttctttggtgccggggcttgtccacagtgccaggccttcgtgcccatcctcaaggacttcttcgtgcggctcacagatgagttctatgtactgcgggcggctcagctggccctggtgtacgtgtcccaggactccacggaggagcagcaggacctgttcctcaaggacatgccaaagaagtggcttttcctgccctttgaggatgatctgaggaggtga

SEQ ID NO: 5 - DNA, 639 bp, Homo sapiens
atggcctccctgttctctggccgcatcctgatccgcaacaatagcgaccaggacgagctggatacggaggctgaggtcagtcgcaggctggagaaccggctggtgctgctgttctttggtgctggggcttgtccacagtgccaggccttcgtgcccatcctcaaggacttcttcgtgcggctcacagatgagttctatgtactgcgggcggctcagctggccctggtgtacgtgtcccaggactccacggaggagcagcaggacctgttcctcaaggacatgccaaagaaatggcttttcctgccctttgaggatgatctgaggagggacctcgggcgccagttctcagtggagcgcctgccggcggtcgtggtgctcaagccggacggggacgtgctcactcgcgacggcgccgacgagatccagcgcctgggcaccgcctgcttcgccaactggcaggaggcggccgaggtgctggaccgcaacttccagctgccagaggacctggaggaccaggagccacggagcctcaccgagtgcctgcgccgccacaagtaccgcgtggaaaaggcggcgcgaggcgggcgcgaccccgggggagggggtggggaggagggcggggccggggggctgttctga

SEQ ID NO: 6 - DNA, 4926 bp, Artificial Sequence
  Synthetic construct 3
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggatgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaactgggcggggagggccttcgtgcgtcgccgcgccgccgtccccttctccatctccagcctcggggctgccgcagggggacggctgccttcgggggggacggggcagggcggggttcggcttctggcgtgtgaccggcggcctctgctaaccatgttcatgccttcttctttttcctacagctcctgggcaacgtgctggttattgtgctgtctcatcattttggcaaagaattgccgccaccatggccagcctcttctccggacgcatcctgattcgcaacaattccgaccaagacgaactggataccgaggccgaagtctcgcggagattggagaacaggcttgtgctgctgttctttggcgcgggagcgtgtcctcagtgccaggctttcgtgccaatcctgaaggatttcttcgtgcggctgactgacgaattctacgtcctccgggccgcccagctggcactggtgtacgtgtcccaagactcaaccgaggaacagcaggatctgttcctcaaggacatgcccaaaaagtggctgttcctgccgtttgaggacgacttgcggcgctagaataaaagatctttattttcattagatctgtgtgttggttttttgtgtgatgtcccttatggtgcttctggctctgcagttattagcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaaccctcaaaatgaaatgaaatcagacccttgtcttacaccataaacaaaaataaatttgaatgggttaaagaattaaactaagacctaaaaccataaaaatttttaaagaaatcaaaagaagaaaattctaatattcacgttgcagccgttttttgaatttgatatgagaagcaaaggcaacaaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccatggcctcactgttctccgggcgcatcctcatccgaaacaacagcgatcaggacgaattggacaccgaggctgaagtctcccgccggctggaaaacaggctcgtgctcctgttcttcggtgccggagcgtgcccgcagtgccaagccttcgtcccaattcttaaggacttctttgtgcgcctcactgatgagttttacgtgctccgggcagcgcagctggccttggtgtatgtgtcgcaagattccactgaggaacaacaggacctgttcctgaaagacatgcctaagaagtggcttttcctgcccttcgaggacgacctgagaagggacctgggacgccagttcagcgtggaacggctgccggccgtcgtggtgctgaagcccgacggggacgtgcttacccgggatggcgctgacgaaatccagaggctgggcaccgcctgtttcgcaaattggcaggaggccgccgaagtgctcgaccggaacttccagctgcccgaggatctggaggaccaggaacctcggtccctgaccgagtgcctcagacgccacaagtaccgcgtggaaaaggccgcgagaggaggacgggacccgggtggcgggggaggcgaagagggcggagccggtggcctgttctgaaacttgtttattgcagcttataatggttacaaataaagcaatagcatcacaaatttcacaaataaagcatttttttcactgcattctagttgtggtttgtccaaactcatcaatgtatcttaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 7 - DNA, 4942 bp, Artificial Sequence
  Synthetic construct 6
gcggccgcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggatgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaactgggcggggagggccttcgtgcgtcgccgcgccgccgtccccttctccatctccagcctcggggctgccgcagggggacggctgccttcgggggggacggggcagggcggggttcggcttctggcgtgtgaccggcggcctctgctaaccatgttcatgccttcttctttttcctacagctcctgggcaacgtgctggttattgtgctgtctcatcattttggcaaagaattgccgccaccatggcctccctgttctctggccgcatcctgatccgcaacaatagcgaccaggacgaactggatacggaggctgaggtcagtcgcaggctggagaaccggctggtgctgctgttctttggtgccggggcttgtccacagtgccaggccttcgtgcccatcctcaaggacttcttcgtgcggctcacagatgagttctatgtactgcgggcggctcagctggccctggtgtacgtgtcccaggactccacggaggagcagcaggacctgttcctcaaggacatgccaaagaagtggcttttcctgccctttgaggatgatctgaggaggtagaataaaagatctttattttcattagatctgtgtgttggttttttgtgtgatgtcccttatggtgcttctggctctgcagttattagcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaaccctcaaaatgaaatgaaatcagacccttgtcttacaccataaacaaaaataaatttgaatgggttaaagaattaaactaagacctaaaaccataaaaatttttaaagaaatcaaaagaagaaaattctaatattcacgttgcagccgttttttgaatttgatatgagaagcaaaggcaacaaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccatggcctccctgttctctggccgcatcctgatccgcaacaatagcgaccaggacgagctggatacggaggctgaggtcagtcgcaggctggagaaccggctggtgctgctgttctttggtgctggggcttgtccacagtgccaggccttcgtgcccatcctcaaggacttcttcgtgcggctcacagatgagttctatgtactgcgggcggctcagctggccctggtgtacgtgtcccaggactccacggaggagcagcaggacctgttcctcaaggacatgccaaagaaatggcttttcctgccctttgaggatgatctgaggagggacctcgggcgccagttctcagtggagcgcctgccggcggtcgtggtgctcaagccggacggggacgtgctcactcgcgacggcgccgacgagatccagcgcctgggcaccgcctgcttcgccaactggcaggaggcggccgaggtgctggaccgcaacttccagctgccagaggacctggaggaccaggagccacggagcctcaccgagtgcctgcgccgccacaagtaccgcgtggaaaaggcggcgcgaggcgggcgcgaccccgggggagggggtggggaggagggcggggccggggggctgttctgaaacttgtttattgcagcttataatggttacaaataaagcaatagcatcacaaatttcacaaataaagcatttttttcactgcattctagttgtggtttgtccaaactcatcaatgtatcttaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcggccgc

SEQ ID NO: 8 - DNA, 4498 bp, Artificial Sequence
  Synthetic construct 7
gcggccgcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcacacaaaaaaccaacacacagatctaatgaaaataaagatcttttattctacctcctcagatcatcctcaaagggcaggaaaagccacttctttggcatgtccttgaggaacaggtcctgctgctcctccgtggagtcctgggacacgtacaccagggccagctgagccgcccgcagtacatagaactcatctgtgagccgcacgaagaagtccttgaggatgggcacgaaggcctggcactgtggacaagccccggcaccaaagaacagcagcaccagccggttctccagcctgcgactgacctcagcctccgtatccagttcgtcctggtcgctattgttgcggatcaggatgcggccagagaacagggaggccatggtggcggcaattctttgccaaaatgatgagacagcacaataaccagcacgttgcccaggagctgtaggaaaaagaagaaggcatgaacatggttagcagaggccgccggtcacacgccagaagccgaaccccgccctgccccgtcccccccgaaggcagccgtccccctgcggcagccccgaggctggagatggagaaggggacggcggcgcggcgacgcacgaaggccctccccgcccagttccttcctgccggcgccgcaccgcttcgcccgcgcccgctagagggggtgcggcggcgcctcccagatttcggctccgccagatttgggacaaaggaagtccctgcgccctctcgcacgattaccataaaaggcaatggctgcggctcgccgcgcctcgacagccgccggcgctccggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcatcccgcccgctcacctgtgggagtaacgcggtcagtcagagccggggcgggcggcgcgaggcggcgcggagcggggcacggggcgaaggcaacgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccgcccccatcgctgcacaaaataattaaaaaataaataaatacaaaattgggggtggggaggggggggagatggggagagtgaagcagaacgtggggctcacctcgatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccatggcctccctgttctctggccgcatcctgatccgcaacaatagcgaccaggacgagctggatacggaggctgaggtcagtcgcaggctggagaaccggctggtgctgctgttctttggtgctggggcttgtccacagtgccaggccttcgtgcccatcctcaaggacttcttcgtgcggctcacagatgagttctatgtactgcgggcggctcagctggccctggtgtacgtgtcccaggactccacggaggagcagcaggacctgttcctcaaggacatgccaaagaaatggcttttcctgccctttgaggatgatctgaggagggacctcgggcgccagttctcagtggagcgcctgccggcggtcgtggtgctcaagccggacggggacgtgctcactcgcgacggcgccgacgagatccagcgcctgggcaccgcctgcttcgccaactggcaggaggcggccgaggtgctggaccgcaacttccagctgccagaggacctggaggaccaggagccacggagcctcaccgagtgcctgcgccgccacaagtaccgcgtggaaaaggcggcgcgaggcgggcgcgaccccgggggagggggtggggaggagggcggggccggggggctgttctgaaacttgtttattgcagcttataatggttacaaataaagcaatagcatcacaaatttcacaaataaagcatttttttcactgcattctagttgtggtttgtccaaactcatcaatgtatcttaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcggccgc

SEQ ID NO: 9 - DNA, 5297 bp, Artificial Sequence
  Synthetic construct 8
gcggccgcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggatgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaactgggcggggagggccttcgtgcgtcgccgcgccgccgtccccttctccatctccagcctcggggctgccgcagggggacggctgccttcgggggggacggggcagggcggggttcggcttctggcgtgtgaccggcggcctctgctaaccatgttcatgccttcttctttttcctacagctcctgggcaacgtgctggttattgtgctgtctcatcattttggcaaagaattgccgccaccatggcctccctgttctctggccgcatcctgatccgcaacaatagcgaccaggacgaactggatacggaggctgaggtcagtcgcaggctggagaaccggctggtgctgctgttctttggtgccggggcttgtccacagtgccaggccttcgtgcccatcctcaaggacttcttcgtgcggctcacagatgagttctatgtactgcgggcggctcagctggccctggtgtacgtgtcccaggactccacggaggagcagcaggacctgttcctcaaggacatgccaaagaagtggcttttcctgccctttgaggatgatctgaggaggtagaataaaagatctttattttcattagatctgtgtgttggttttttgtgtgatgtcccttatggtgcttctggctctgcagttattagcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaaccctcaaaatgaaatgaaatcagacccttgtcttacaccataaacaaaaataaatttgaatgggttaaagaattaaactaagacctaaaaccataaaaatttttaaagaaatcaaaagaagaaaattctaatattcacgttgcagccgttttttgaatttgatatgagaagcaaaggcaacaaaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccaccctcagaacagccccccggccccgccctcctccccaccccctcccccggggtcgcgcccgcctcgcgccgccttttccacgcggtacttgtggcggcgcaggcactcggtgaggctccgtggctcctggtcctccaggtcctctggcagctggaagttgcggtccagcacctcggccgcctcctgccagttggcgaagcaggcggtgcccaggcgctggatctcgtcggcgccgtcgcgagtgagcacgtccccgtccggcttgagcaccacgaccgccggcaggcgctccactgagaactggcgcccgaggtccctcctcagatcatcctcaaagggcaggaaaagccatttctttggcatgtccttgaggaacaggtcctgctgctcctccgtggagtcctgggacacgtacaccagggccagctgagccgcccgcagtacatagaactcatctgtgagccgcacgaagaagtccttgaggatgggcacgaaggcctggcactgtggacaagccccagcaccaaagaacagcagcaccagccggttctccagcctgcgactgacctcagcctccgtatccagctcgtcctggtcgctattgttgcggatcaggatgcggccagagaacagggaggccatggctatggaaagccctgtccccgacggcagccggccctggcgcatcacctgagggtcacggcgctttatactggcctgtgggatcagcccgggcctcttcaccttaaaagctcccaaaccctacactacctgatctcttaattgggcctgggacttagacctcctcctcccacctcttgctcccctgctcctgaggcgagcccgcaagggcggctcagagggatttgctgggaaccaaactcagacgccccacccatccccgccaggaaggtggcggtgactgtgggagccgctgtggccctctctgtgtttatgtgttctctgttcccttctactgtcctgcgtccgcctttggggcaggtggacccacgggtcaggatttgcagactggggggtgggtggtgaaactgctcagagcccgaatcctcctttctcatgtggttctcttgaaagcccagggagactatttagggccgaggcctggaaccggatccaagaatgtgagacccagaagtgagcctcctctgagatcacctgccccaccgggtcccttctgagggtgggatggctggccagccttgtcccattaccgccatgagctggctgggtggccttgaacagggcgacctctccctcttgaccgagattccccttctgtctagagggagataagcaactctgaggcttccctgggcatccaaggccaccatgctatttggaagccctttgagaacacgaagtgccctatgaagtgggggtccagcagaggaggagtggggtgtctgggggaagcccccagccttgcctgagccccggagctggccctccgctcgccctgtctgcccttgtccaggattacgctaatgacccagctcagtgcaatcccaccaatgccctgggccacacgtgtgccagcccccacatcccaggaaggagagtggaggtggcagaggtgggaggaggcctggaaaggcggtaggttctgcaccttcaccggctctgctaattgatgattagggctaacagaagatcaagtcccattttagcctaaagcagattactgtcctagaaagtcactcagcacctgtgggggcacgtgtgtccccaggccaggtgaaaactcatttgtggtcccacactcttgcagaagggtggatgaaagtggagggagcactagagaaggatatgaagaacgagggggtgagggggtgggatggggggagaggagcgggagcagtggcggcccaagaaagccccaaggaaagagggcccctggggaaaggccagagggacccaagccaggccgggccccaatgtcagcgccctctgctctgcccggagtctcccaccctcgctgccagctggggccctggccagggtggaagattagatgttggacttgtctctaatgcgactactcctaaatgactccacgcgggagagccgattaggagttatgggggctgctgtcatcttgactcaggggctttgtcactaacagccgatggctgctacagctataggacaaggaaccatttggccatttgcctttgacactttaaaaagatcgtggtaaaatatacataatacaaagtgtaccatcgctaccgtgtgtaagtgtatagtccagtcaccaccatccgtctccaaactcttttcattgcatgaaacggaagctctgtcctcatgaaacacccatgccctttccccacccctcagcctccaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcggccgc

SEQ ID NO: 10 - DNA, 4360 bp, Homo sapiens
tcaacaaatgtctcacaattaatacatttggcctaatgttttaggacaagacaaatatttaatttgaacaagagatgtaaaagttaggaattactctagtgaaaacagatttggatgatgatggacaatattagtgtggtgcctcaacaaagacttttgcctcttgcatactattatgattgcacggtaatcttacccagctagtgttttcttaatgtcctggggagcctgatcattgtaaattacctgtgacatctcattaatcctctttttcctcacaaacgcataattaatggtaacattgacaataatacacaaattagctaagtgacgctttatggtgttggttttgattaacgtcaggaacccaaaaatgacgttaatgtaatgcagatcccaaaaggcagtctgcactttgaccccagacatacattttgtttgcaccatcaccaagattaatcctcctctggatgaagatgagacaaaactctggccactggcagtgtgtgctaggctgtcggaaaacaaaccgcctcaactgcatttgtttaagaaggaaatcttaatgcacagaaaacaacggcgtagtgtcatttcctattgtctagttaggacaaagataaatgagcaactgtcatgattcagatcaactgcttccctgctaacttctattatttgcttatttatggactggtgtttaattaaaagtatacctagatatataaaccacatgataaaataattgaaattccttttgctttcactataaatggtttttggaaatcaagactgatttttatatgcatttcaacctgcaggacctttaaatgataaattcttattaaccacaagagggtaaacaaaatcaatgcctgagaaaatatgctttgtctctgacttgagtaacaaagtaaaaacactagaagaaatttggataaagttcttaacttcatcagtgaggcatccttgtagactctgagaaagctgatatagagacgctttttccaaattataaaagagacaaacactttaagtaagtcacagtagcatctactgctgcatcatcaacttctaaattcaggcttaatcatgtgactttaatataaacatgaacagtttcttaattacgattttcacaaatctgagttaagtactgaatctttttaacactccaaagaggtagcatatgctaattttttaaagatgcaaccttctattttcttcgtaacaattacttatagctgaaacatcttaatggtatacattttttaaaaataatagtctcgtatttgattaatttaacatctacttatttaggctatttttcaaaatgttactgctactaaaccacagccctggctaatgatcgtattccacttcaaaaacggagtaagaagaaaagccatattttcatgctttgttctatttttcacacaaataactatcatttatggacaaaaatatgcaacacgtaaagtgatgtcataaaaatttgaaataaatccaccaaaattggaaagcaaagatcaccgaaattatgaaacattgtgggtatatttgataaaaagaagatactatttttgattaaaaaaagagaatatcttaaaaagaagattttggttgctgtgggtatgtattttcaattgtgaggcctctaatccacctctcccccaaaataagaaaacagactgccttacaactgcttgtcatagcagtttaattttgaaaaatgcctcatctgtccctgcttgcaatatattgtcttacgactacgtttaccttttttattttttgggggggggttattgaaagttacaaggagcaaaataatgtttttagagaatttttttcttctcttgtttaaatattattttaaaatcaaaagcagctcatttttaaatcgacatattaagtgttagctgcaccaactttcaccagcacacaaacccattgtaaagcaaactggaatttaaggggcaatggggagtcctacagaggtcatattttcactctgcctaactaaaccataaatcaagattacaacaataaattattttttattattttggcaggaataagtatagccttgaattttgaaagatatttgggcttaattcatatgttaaataggtcgggataaaccaatgctagaagaaattgaaactggcccaatattatacagaaacccaaattaggcactcaccaccaaactaattagtaattgataaatttacaatagtaatgtaatttctttacactgcaaagccaaagaaataggataattgaaagcttcaaatctaaaaaaaaaaaaattgacaatcttttaatcaaaggcattgtttacaacgtggcttttaggtagctgcagtaacatctttaactggttaaaaacactcatcaagctataaaactactttcttagatgtctatagcaaactgtttgaaacctcccaaagtgccttaatttatattttttcagcagctacaatgaatgttactttcttccagtccaaatgtatcattttcacttttcaacttattcaagttcatagagagcaacctatgaagccacaattttatagaaatctatttttcaggtttaaatgtggtaccctgacttttcataagctccaatagttgtgccaagttacttttttgttgttgttctaaggaaagattttatgcaaataaagagactgctctgtgggaaagcatgttttcttaagtatttaatcggacatttccacaccacaacactttgtagacatgtttttgctttgaaaacataatcttatcaggggtggttggtgtacaattcttgtctgttttcttacctttgaatttgagctctatttataaggctcaactaaatgatgtctgcagctaaggcatttccttactagggagttttacacgaacacaattaatatgaaagcacacaacgcattgaacagggtgtcaatttaggtttccggtacaatgtaaccttatcgaagtgttcccgtttacatagaagaaaacaaatatgataaagcggttaaatatgtatctgtcttagatttctaacattttcctctcgatatatcttttcggaataaacaaagggaagtgaattcttcattccttatttgtacaaacaagtgaaattgggtccccttcacgatggaaggcactggcctttctcctcacatggaacaaactgctagagaaacttggtttacacaaaatggaaacatttaatccttgcatctatatttcaaaatagtacatggtttttccataaatctatgtgtgatgacatatttcgtaacattacataaggctttccatgcatttttattcatgggagaatggcttgcagaaaatcacaacattgtactatgcttctccagcaagttaattttcactttactgcgaatatacagtttatggaaaaataatagttatttaagtgtagcgtacttacctgttgctgtgggaaaattcctaccattaaaaatgtgctttcacttcagttattattataatgtaggctataccaatgcctgatgatttttataaacagaacctataaatctgcattttggttcagggctgtactatttagacaaatgaaaatgaagcagcatgacatgaagtccaaaggaacgttcttatacatgcctcaactttggttttttggtaactgccctttggagactatcctttattaaggatcttaatgagggttctgcctgtatttttctcccaagaatgtcaaataaggttcgaaagcaaatgtaaactgtaatacaagtgacacacactaaaaatagccctataaatctgttcattttgtttagatagtctccagagttgtaaaaagtaggactgttcagctttattatttgttttggaaaacctaattctgtagagaataggccactgataatgctatattactttagaggagatttattaagactgtctctagatttaatttttttaaaataaagaaactctttcctgtttttattttccaaataatctacaagataattaactgaatataatctataatattaaataaattttcccatttactcattagaattaagattttgaaaggcacatttatctaaactaaaattaattaaaatttcaaatagaattctcattcctattaacctgtcaaatatgttgcgttcaatgtttccagtaacagaacaacaatatttattatctaacgtaccataagtttttaaaaataactccaggagaacatttttggaacacttacaatctgaatagacaactaaaaaaatcagacatttctgtgtgaaaaatagctattgtaaattaaaatggttgagggagaata

SEQ ID NO: 11 - DNA, 330 bp, Homo sapiens
atggcctccttgttctccggccgaatcctgatccgaaacaatagtgaccaggatgagctggatacagaagctgaggtcagtcgcagactggagaacaggctggtccttctgttctttggtgctggggcttgtcctcagtgccaggccttcgtgcccatcctcaaggacttctttgtgcggctcacggatgagttctatgtactgcgggcagctcaactggctctagtatatgtgtcacaggactccactgaggagcagcaggacctcttcctgaaggacatgccaaagaaatggcttttcctgccctttgaggatgatctgaggaggtag

SEQ ID NO: 12 - DNA, 330 bp, Artificial Sequence
  Synthetic codon-optimized
atggccagcctcttctccggacgcatcctgattcgcaacaattccgaccaagacgaactggataccgaggccgaagtctcgcggagattggagaacaggcttgtgctgctgttctttggcgcgggagcgtgtcctcagtgccaggctttcgtgccaatcctgaaggatttcttcgtgcggctgactgacgaattctacgtcctccgggccgcccagctggcactggtgtacgtgtcccaagactcaaccgaggaacagcaggatctgttcctcaaggacatgcccaaaaagtggctgttcctgccgtttgaggacgacttgcggcgctag

SEQ ID NO: 13 - DNA, 639 bp, Artificial Sequence
  Synthetic codon-optimized
atggcctcactgttctccgggcgcatcctcatccgaaacaacagcgatcaggacgaattggacaccgaggctgaagtctcccgccggctggaaaacaggctcgtgctcctgttcttcggtgccggagcgtgcccgcagtgccaagccttcgtcccaattcttaaggacttctttgtgcgcctcactgatgagttttacgtgctccgggcagcgcagctggccttggtgtatgtgtcgcaagattccactgaggaacaacaggacctgttcctgaaagacatgcctaagaagtggcttttcctgcccttcgaggacgacctgagaagggacctgggacgccagttcagcgtggaacggctgccggccgtcgtggtgctgaagcccgacggggacgtgcttacccgggatggcgctgacgaaatccagaggctgggcaccgcctgtttcgcaaattggcaggaggccgccgaagtgctcgaccggaacttccagctgcccgaggatctggaggaccaggaacctcggtccctgaccgagtgcctcagacgccacaagtaccgcgtggaaaaggccgcgagaggaggacgggacccgggtggcgggggaggcgaagagggcggagccggtggcctgttctga

SEQ ID NO: 14 - DNA, 4934 bp, Artificial Sequence
  Synthetic construct 11
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggatgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaactgggcggggagggccttcgtgcgtcgccgcgccgccgtccccttctccatctccagcctcggggctgccgcagggggacggctgccttcgggggggacggggcagggcggggttcggcttctggcgtgtgaccggcggcctctgctaaccatgttcatgccttcttctttttcctacagctcctgggcaacgtgctggttattgtgctgtctcatcattttggcaaagaattgccgccaccatggcctccttgttctccggccgaatcctgatccgaaacaatagtgaccaggatgagctggatacagaagctgaggtcagtcgcagactggagaacaggctggtccttctgttctttggtgctggggcttgtcctcagtgccaggccttcgtgcccatcctcaaggacttctttgtgcggctcacggatgagttctatgtactgcgggcagctcaactggctctagtatatgtgtcacaggactccactgaggagcagcaggacctcttcctgaaggacatgccaaagaaatggcttttcctgccctttgaggatgatctgaggaggtagaataaaagatctttattttcattagatctgtgtgttggttttttgtgtgatgtcccttatggtgcttctggctctgcagttattagcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaaccctcaaaatgaaatgaaatcagacccttgtcttacaccataaacaaaaataaatttgaatgggttaaagaattaaactaagacctaaaaccataaaaatttttaaagaaatcaaaagaagaaaattctaatattcacgttgcagccgttttttgaatttgatatgagaagcaaaggcaacaaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccatggcctccctgttctctggccgcatcctgatccgcaacaatagcgaccaggacgagctggatacggaggctgaggtcagtcgcaggctggagaaccggctggtgctgctgttctttggtgctggggcttgtccacagtgccaggccttcgtgcccatcctcaaggacttcttcgtgcggctcacagatgagttctatgtactgcgggcggctcagctggccctggtgtacgtgtcccaggactccacggaggagcagcaggacctgttcctcaaggacatgccaaagaaatggcttttcctgccctttgaggatgatctgaggagggacctcgggcgccagttctcagtggagcgcctgccggcggtcgtggtgctcaagccggacggggacgtgctcactcgcgacggcgccgacgagatccagcgcctgggcaccgcctgcttcgccaactggcaggaggcggccgaggtgctggaccgcaacttccagctgccagaggacctggaggaccaggagccacggagcctcaccgagtgcctgcgccgccacaagtaccgcgtggaaaaggcggcgcgaggcgggcgcgaccccgggggagggggtggggaggagggcggggccggggggctgttctgaaacttgtttattgcagcttataatggttacaaataaagcaatagcatcacaaatttcacaaataaagcatttttttcactgcattctagttgtggtttgtccaaactcatcaatgtatcttaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcggccgc

SEQ ID NO: 15 - DNA, 12646 bp, artificial sequence
  Synthetic CT35
tagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgccaccatggccagcctcttctccggacgcatcctgattcgcaacaattccgaccaagacgaactggataccgaggccgaagtctcgcggagattggagaacaggcttgtgctgctgttctttggcgcgggagcgtgtcctcagtgccaggctttcgtgccaatcctgaaggatttcttcgtgcggctgactgacgaattctacgtcctccgggccgcccagctggcactggtgtacgtgtcccaagactcaaccgaggaacagcaggatctgttcctcaaggacatgcccaaaaagtggctgttcctgccgtttgaggacgacttgcggcgctagtgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggatccaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccaggaagctttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggatgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactaggccatacaggccgccaccatggcctcactgttctccgggcgcatcctcatccgaaacaacagcgatcaggacgaattggacaccgaggctgaagtctcccgccggctggaaaacaggctcgtgctcctgttcttcggtgccggagcgtgcccgcagtgccaagccttcgtcccaattcttaaggacttctttgtgcgcctcactgatgagttttacgtgctccgggcagcgcagctggccttggtgtatgtgtcgcaagattccactgaggaacaacaggacctgttcctgaaagacatgcctaagaagtggcttttcctgcccttcgaggacgacctgagaagggacctgggacgccagttcagcgtggaacggctgccggccgtcgtggtgctgaagcccgacggggacgtgcttacccgggatggcgctgacgaaatccagaggctgggcaccgcctgtttcgcaaattggcaggaggccgccgaagtgctcgaccggaacttccagctgcccgaggatctggaggaccaggaacctcggtccctgaccgagtgcctcagacgccacaagtaccgcgtggaaaaggccgcgagaggaggacgggacccgggtggcgggggaggcgaagagggcggagccggtggcctgttctgatagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaaacatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggctaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgct

SEQ ID NO: 16 - DNA, 12451 bp, artificial sequence
  Synthetic CT37
tagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgccaccatggcctccctgttctctggccgcatcctgatccgcaacaatagcgaccaggacgagctggatacggaggctgaggtcagtcgcaggctggagaaccggctggtgctgctgttctttggtgctggggcttgtccacagtgccaggccttcgtgcccatcctcaaggacttcttcgtgcggctcacagatgagttctatgtactgcgggcggctcagctggccctggtgtacgtgtcccaggactccacggaggagcagcaggacctgttcctcaaggacatgccaaagaaatggcttttcctgccctttgaggatgatctgaggaggtgatcatctcatggatccaagagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgaaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttactagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgcgctgccttcgccccgtgccccgctccgccgccgcctcgcgccgcccgccccggctctgactgaccgcgttactcccacaggtgagcgggcgggatgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgccaccatggcctccctgttctctggccgcatcctgatccgcaacaatagcgaccaggacgagctggatacggaggctgaggtcagtcgcaggctggagaaccggctggtgctgctgttctttggtgctggggcttgtccacagtgccaggccttcgtgcccatcctcaaggacttcttcgtgcggctcacagatgagttctatgtactgcgggcggctcagctggccctggtgtacgtgtcccaggactccacggaggagcagcaggacctgttcctcaaggacatgccaaagaaatggcttttcctgccctttgaggatgatctgaggaggtgatcatctcatggatccaagagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaaacatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggctaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgct